(19) United States
(12) Patent Application Publication
(10) Pub. No.: US 2011/0223587 A1
(43) Pub. Date: Sep. 15, 2011

(54) OPTICAL PARTICLE CHARACTERIZATION SYSTEM

(76) Inventors: Joseph D. SCHULMAN, Palm Beach, FL (US); Dixie King, Palm Beach, FL (US)

(21) Appl. No.: 12/722,352

(22) Filed: Mar. 11, 2010

(51) Int. Cl. C12Q 1/68; G01N 21/47; G01N 21/64; G01N 21/63; G06F 7/00; B07B 13/00

(52) U.S. Cl. 435/6.1; 356/338; 250/458.1; 700/223; 250/574; 422/404

(57) ABSTRACT

A particle characterization system including a flow source to produce a stream of particles; a source of light or other energy directed at the stream of particles to cause fluorescence or scattered light to be emitted from the particles; a detector of the fluorescent or scattered light including a plurality of light receivers in an enclosed three-dimensional arrangement to detect the light emitted by the particles; and a computer which receives information from the detector regarding light emitted by a particle in response to the source of light or other energy, the computer programmed to determine a characteristic of the particle based on the light collected from the particle.

### BACKGROUND OF THE INVENTION

1. Field of the Invention

This invention relates to a system optically characterizing particles.

2. Background

The disclosed subject matter relates to optically characterizing particles. In one embodiment, such characterization may be employed in a technique for enriching a sperm sample from a male. The goal is to increase the proportion of X-bearing or Y-bearing sperm cells by sorting sperm based on differences in DNA content. This enriched sperm sample can be used to fertilize the partner's eggs by intrauterine insemination (IUI), in vitro fertilization (IVF), or intracytoplasmic sperm injection (ICSI), by way of example.

Human cells normally possess 46 chromosomes comprising 22 pairs of autosomes (numbered 1 to 22) and 2 sex chromosomes (X and Y). Gametes (egg and sperm) each contribute one-half of the genetic material, or one member of each of the autosome pairs and one sex chromosome, to the embryo. Each gamete contains 23 chromosomes. The process, which results in gametes having half the genetic material of the somatic cells, meiosis, results in half of the sperm cells bearing an X chromosome, and the other half bearing a Y chromosome. The X chromosome is significantly larger than the Y chromosome, such that the total DNA content of an X chromosome-bearing human sperm cell is approximately 2.4% greater than that of a Y chromosome-bearing human sperm cell. A conventional technique for enriching a sperm sample comprises obtaining a sperm sample comprising sperm cells from a mammalian male; contacting the sperm cells with a detectable DNA-interacting agent that imparts to a sperm cell the ability to emit a light signal having an intensity proportional to the amount of DNA present in the sperm cell; separating those sperm cells based upon a measured signal intensity, and collecting the separated sperm cells to obtain an enriched sperm sample. In one embodiment, the agent is Hoechst 33342. In another embodiment, the separating is carried out by flow cytometry. In yet another embodiment, the flow cytometry is fluorescence activated cell sorting.

Conventionally, sorting sperm cells into X- and Y-bearing populations has been accomplished by a technique known as flow cytometry. Other techniques have been employed for sorting sperm populations based on size, mass, or density. Flow cytometry-based techniques exploit the difference in size between X (larger) and Y (smaller) chromosomes. For example, in bovine sperm, an X-chromosome bearing sperm cell contains 4 percent more DNA than its Y-chromosome bearing counterpart. To detect this difference in DNA content, the sperm cells are stained with a flourochrome that binds to the sperm DNA, and made to flow individually before an irradiating laser and a detector which measures the amount of fluorescence of each cell. Based on the amount of detected fluorescence, individual sperm are classified into three categories: X-bearing, Y-bearing, and indeterminate. However, because of difficulties primarily associated with the orientation of individual cells as they pass through the detector, a substantial number of the sperm cells are characterized as indeterminate. Additionally, because of the minor difference in DNA content between the between the X- and Y-bearing sperm cells, a certain portion is misidentified (e.g., an X-chromosome bearing cell is identified as a Y-chromosome bearing cell).

U.S. Pat. No. 7,371,517, which is herein incorporated by reference in its entirety, describes a conventional flow cytometry apparatus for sorting sperm cells. With reference to U.S. Pat. No. 7,371,517, FIG. 1, a supply of sperm cells is output via a nozzle after being surrounded by a sheathing fluid. Ideally, sperm cells exit serially with a specific orientation (as discussed below, the orientation of the cell affects the amount of detected fluorescence). A piezoelectric or ultrasonic actuator vibrates the exit stream into droplets, each nominally containing no more than a single sperm cell. The stream of sperm cells passes through a detector. Based on the detected fluorescence, each cell is categorized by a processor. Based upon the categorization of the sperm contained within a droplet, each droplet is electrostatically deflected to a cell collection vessel.

U.S. Pat. No. 7,371,517, FIG. 16 shows a conventional detector in more detail. As shown, the detector uses an irradiating laser at a first wavelength to cause the flourochrome bound to DNA in a sperm cell to fluoresce. Two photodetectors are employed: one at 0 degrees (opposite the laser), and the other at 90 degrees. Filters may be used to screen out laser illumination. Photomultipliers are typically used as photodetectors. Based upon the output of the two photodetectors, a categorization of the cell is preformed. For example, the output of the 90 degree detector is used to determine the quality of the detected signal. If the signal is too weak, it is likely there sperm cell was not properly irradiated (typically due to the orientation of the cell), and the signal is considered unreliable for determining whether the cell bears an X or Y chromosome. However, if the signal at the 90 degree detector is satisfactory, the signal received by the 0 degree detector for that same sperm cell is used to characterize a cell as X or Y chromosome bearing. If the signal at the 90 degree detector is not satisfactory, it is typically because the sperm cell is not oriented properly with respect to the 90 and 0 degree detectors. U.S. Pat. No. 7,371,517, FIG. 3B illustrates a histogram of signals received at the 0 degree detector. As is shown, the result is a bimodal histogram, with the left peak associated with Y chromosome-bearing cells, and the right peak associated with X chromosome-bearing cells. FIGS. 4-10 of the above patent show two-dimensional plots obtained by observations with both the 0 and 90 degree detectors.

The orientation of the sperm cell has a significant effect on the amount of detected fluorescence. As roughly illustrated in U.S. Pat. No. 7,371,517, many sperm cells have a flat, coin-like shape. Because of this shape, detected fluorescence is sensitive to the orientation of the cell to the photodetector. Fluorescence is greatest when the sperm cell is oriented edgewise relative to the photodetector. Accordingly, as shown in FIG. 16 of the above patent, conventionally it is attempted to irradiate the cells on a flat side, and the 90 degree detector is situated so as to observe the edgewise fluorescence. Variation in orientation of the sperm cell may result in approximately a 2× variation in detected fluorescence—in other words, the intensity of the fluorescence signal of misoriented cells is only about half that of properly oriented cells. This variation in detected fluorescence is far greater than the difference of approximately 4% which must be detected in order to effectively sort sperm cells into X- and Y-bearing populations.

Variations in orientation of sperm cells lead to two primary issues: (1) inability to categorize cells, generally due to too little detected fluorescence, and (2) miscategorization of cells (biased toward miscategorizing X cells as Y cells, as a minor decrease in the detected intensity causes an X cell to resemble a Y cell). Much effort has been undertaken in the field to cause sperm cells to pass through the detector with a consistent orientation, to better ensure that the 90 degree detector “views” cells edgewise and the flat side of the sperm head is oriented towards the 0 degree detector. Although many improvements have been made with respect to this aspect, a substantial number of cells still fail to be characterized, primarily due to misorientation of cells as they pass through the detector.

All publications, scientific, patent or otherwise discussed in this application are hereby incorporated by reference in their entirety for all purposes, including Martin et al., Clinical Genetics 1995: 47: 42-46. Escudero et al., Prenatal Diagnosis 2002: 20: 599-602. Munné et al., Reproductive Biomedicine Online (RBM Online) 2002a: vol. 4. no. 2. 183-196. Munné et al., Abstract Presented at the 58th Annual Meeting of the American Society for Reproductive Medicine (ASRM), 2002, Seattle, Wash.; and Daniel, Human Genet 51; 171-182:1979.

### SUMMARY OF THE INVENTION

One aspect of the invention relates to a particle characterization system including a flow source to produce a stream of particles; a source of light or other energy directed at the stream of particles to cause fluorescence or scattered light to be emitted from the particles; a detector of the fluorescent or scattered light including a plurality of light receivers in an enclosed three-dimensional arrangement to detect the light emitted by the particles; and a computer which receives information from the detector regarding light emitted by a particle in response to the source of light or other energy, the computer programmed to determine a characteristic of the particle based on the light collected from the particle.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. The present invention may be practiced without some or all of these specific details. In other instances, well known process operations have not been described in detail, in order not to unnecessarily obscure the present invention. Accordingly, the drawings and description are to be regarded as illustrative in nature, and not as restrictive.

Although specific examples of the disclosed subject matter are provided that relate to flow cytometry, the subject matter of this application is not limited to the described uses in the field of flow cytometry, as the disclosed techniques are effective for characterizing many types of particles, whether natural or manufactured. The disclosed techniques are particularly useful for characterizing nonsymmetric particles, including genes, chromosomes, and proteins. Additionally, there are many nanotechnological materials in which the product, or an intermediate product, is a particulate which one may wish to characterize for the purposes of quality control or product sorting.

### BRIEF DESCRIPTION OF THE DRAWINGS

FIG. 1 illustrates a cross-section of an embodiment of the disclosed subject matter.

FIG. 2A illustrates an embodiment of the disclosed subject matter in which the light gathered by the light transmitting conduits is directed to a single photodetector.

FIG. 2B illustrates an embodiment of the disclosed subject matter in which each light transmitting conduit transmits the light it receives to a corresponding photodetector, and the photodetectors are included in an array.

FIG. 2C illustrates an embodiment of the disclosed subject matter in which photodetectors are provided in a plurality of arrays, and a plurality of lasers are employed to irradiate a particle.

FIG. 2D illustrates an embodiment of the disclosed subject matter in which an optical multiplexer is used to selectively provide light transmitted by the light transmitting conduits to a number of photodetectors less than the number of light transmitting conduits.

FIG. 3 illustrates a simplified side view of an embodiment of the disclosed subject matter, in which the light-receiving ends of the light receivers are arranged in a spherical arrangement about the point of illumination of a stream of particles being characterized.

FIGS. 4A, 4B, and 4C illustrate various example configurations for the light-receiving ends of the light receivers.

The drawings disclose illustrative embodiments, and do not set forth all embodiments of the invention. Other embodiments may be used in addition or instead. Details that may be apparent or unnecessary may be omitted to save space or for more effective illustration. Conversely, some embodiments may be practiced without all of the details that are disclosed. When the same numeral appears in different drawings, it is intended to refer to the same or like components or steps.

### DETAILED DESCRIPTION OF THE DISCLOSED SUBJECT MATTER

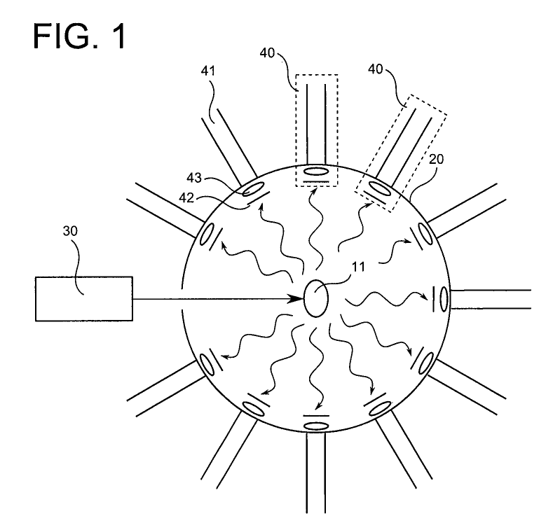

FIG. 1 illustrates a cross sectional view, viewed from above, of a portion of an embodiment of a particle characterization system according to the techniques described in this application. A stream of particles, including illustrated particle 11, passes through detector 20 for characterization by the particle characterization system. FIG. 3A illustrates a stream of particles 11 provided by flow source 10. In the embodiment illustrated in FIG. 1, a particle 11, such as a sperm cell, passes through detector 20. Other types of particles that may be characterized include, but not limited to, individual molecules, aggregates of molecules, crystals, viruses, prions, pollen, zooplankton, single cell organisms (including but not limited to bacteria and protozoa), single cells, and aggregates of cells (from larger organisms, including fish, humans, other animals, and plants). Sperm cell size varies by species. A human sperm head is about 5 microns long and about 3 microns wide, and overall length of the sperm cell, including the tail, is about 60 microns. A bovine sperm head is about 10 microns long and about 5 microns wide, and overall length of the sperm cell, including the tail, is about 50 microns. A pig sperm head is about 8 microns long and about 4 microns wide, and overall length of the sperm cell, including the tail, is about 40 microns. A horse sperm head is about 7 microns long and about 3 microns wide, and overall length of the sperm cell, including the tail, is about 60 microns. A human sperm cell has a head with dimensions of approximately 6 microns across by 10 microns long, with a tail of about 50 microns in length. In some embodiments the particles being characterized are up to approximately 250 microns in size. In the illustrated embodiment, the particles pass downward through detector 20 by gravity. However, one skilled in the art would appreciate that other techniques may be employed for causing the particle to move through the detector, including, but not limited to, hydraulic or pneumatic pressure, electrical or magnetic charge, or suspended in a liquid and traveling through a tube, pipe, or cuvet. In an example embodiment in which the particles are sperm cells, the particles are entrained in a liquid, which is useful in maintaining the viability of the sperm cells and better enables the flow source to provide a stream of cells for characterization. In some embodiments, a particle 11 may be immobile, with detector 20 positioned relative to particle 11 in order to perform characterization of particle 11.

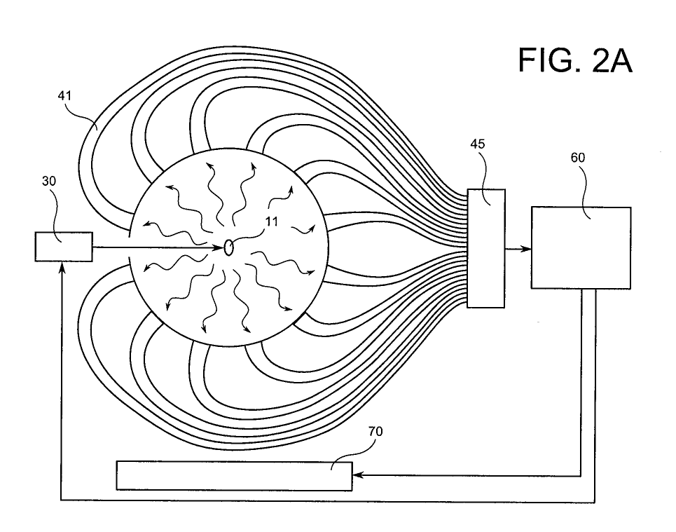

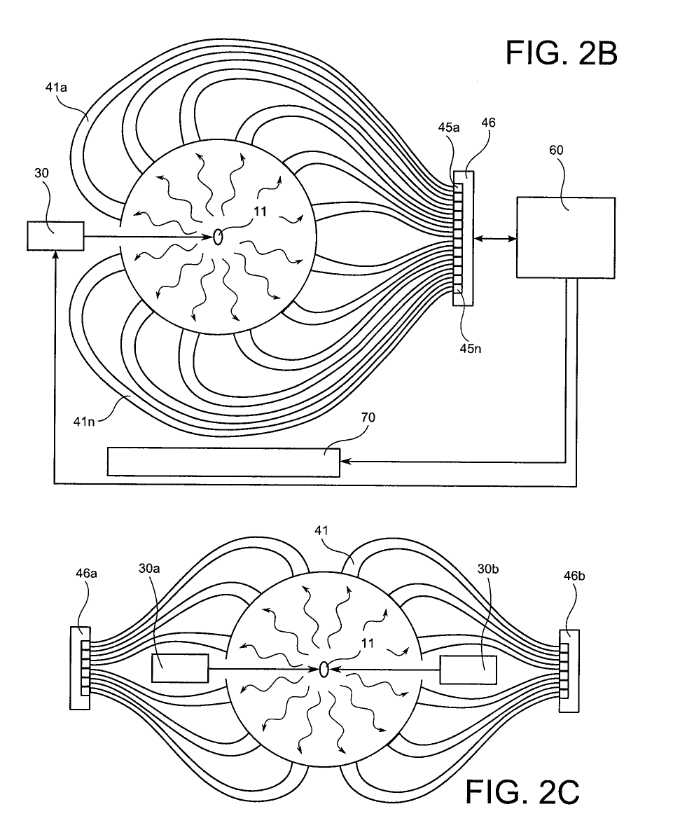

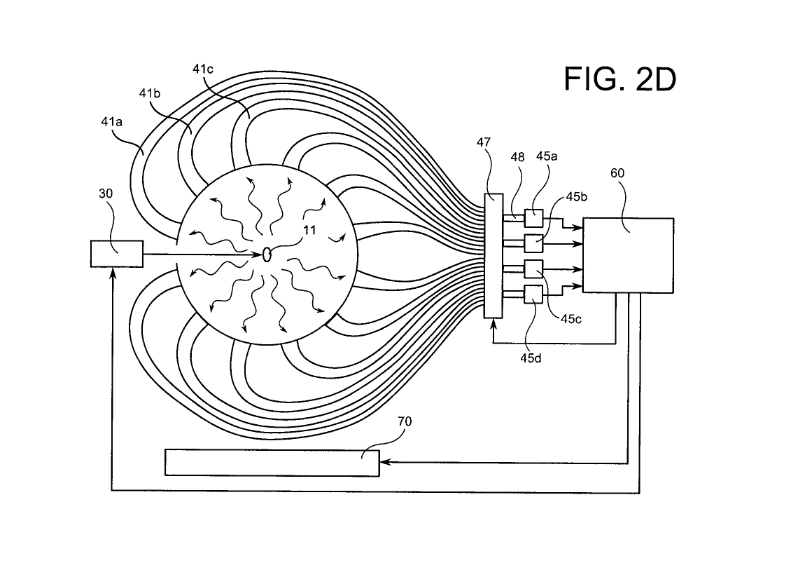

As particle 11 passes through detector 20, a source of light or other energy directed at the stream of particles causes fluorescence or scattered light to be emitted from the particles. For example, in the embodiment illustrated in FIG. 1, a laser 30 irradiates sperm cell 11. Prior to passing through detector 20, sperm cell 11 was treated with a flourochromatic stain that binds to DNA contained in sperm cell 11. When irradiated by laser 30, the flourochromatic stain in sperm cell 11 fluoresces, with an intensity that corresponds to the amount of DNA in sperm cell 11. Depending on the nature of particle 11 and the characteristics one wishes to determine with respect to particle 11, laser 30 may alternatively be any source of light (coherent or noncoherent) or other energy directed at the path of the stream of particles 11 passing through detector 20. More than one laser 30 may be used to illuminate particle 11, as illustrated in FIG. 2C.

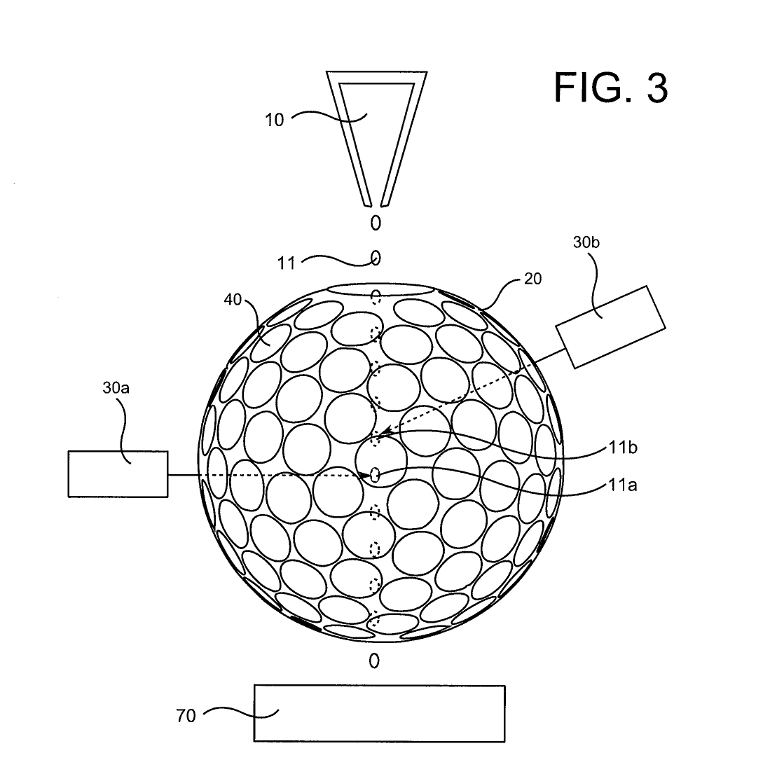

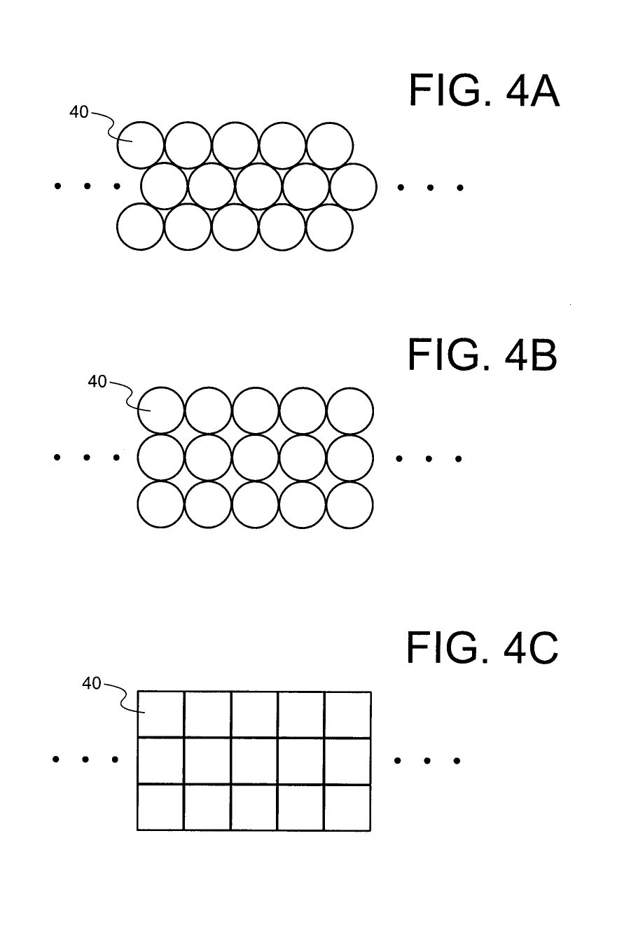

Detector 20 comprises a plurality of light receivers 40 arranged such that a light receiving end of each of the light receivers 40 receives fluorescent or scattered light emitted by particle 11. Accordingly, each of light receivers 40 detects a portion of the light emitted by particle 11. As illustrated in FIGS. 1 and 2A-2D, in some embodiments, each of light receivers 40 may include a light-transmitting conduit 41, such as an optical fiber, liquid waveguide, or nanofiber, although other light-transmitting conduits may be employed. In such embodiments, the light received by light receivers 40 is conducted to one or more photodetectors (not illustrated in FIG. 1). In some embodiments, light receivers 40 may instead be photodetectors disposed directly around the position where particle 11 emits light, without an intervening light-transmitting conduit. For example, photodiodes may be disposed around particle 11 to directly capture light emitted by particle 11 when it is irradiated by laser 30. However, depending on the type of photodetectors employed, it is often easier to more densely pack the light receiving ends of the light-transmitting conduits 41 than to densely pack photodetectors. Also, although FIGS. 1 and 2A-2D illustrate a relatively sparse arrangement of light receivers 40, in many embodiments the light receivers are closely packed, as illustrated in FIGS. 4A-4C, in part to maximize the light receiving area of detector 20. Additionally, many embodiments include a much greater number of light receivers than is illustrated in the drawings of this application. For the convenience of illustration, a small number of light receivers are illustrated in each of the drawings. However, many embodiments may have hundreds, thousands, or even millions of light receivers, depending upon the benefits that may be obtained by having an increased number of light receivers for a given application.

In some embodiments light receiver 40 includes a filter 42 disposed between particle 11 and light-transmitting conduit 41. For example, a long pass filter might be employed to allow fluorescent light to pass through to light-transmitting conduit 41, but not light scattered by particle 11 or emitted by laser 30. Alternatively or in addition to filters 41, prisms may be used to separate light emitted by particle 11 by wavelength. Although FIG. 1 illustrates filter 42 disposed between particle 11 and light-transmitting conduit 41, filter 42 may be disposed anywhere along the optical path between particle 11 and a photodetector 45 (not illustrated in FIG. 1) which measures the light emitted by particle 11. Also, light-transmitting conduit 41 may itself function as a filter. Different types of filters may be employed for various light receivers 40. For example, one light receiver 40 may include a low-pass filter, whereas a neighboring light receiver 40 may include a high-pass filter. Accordingly the two light receivers 40 could be employed to determine different characteristics of light emitted by particle 11.

Additionally, in some embodiments light receiver 40 includes a lens 43 disposed between particle 11 and light-transmitting conduit 41. In some embodiments, lens 42 may be formed directly on light receiver 40. For example, where a light receiver 40 includes an optical fiber as a light-transmitting conduit 41 for conducting light emitted by particle 11, the end of the optical fiber may be subjected to heat and/or abrasive polishing in order to provide a lens 43 that aids the capture of light emitted by particle 11. Also, optical interfaces in light receiver 40 may have anti-reflection coatings in order to increase the amount of conveyed light.

In some embodiments, a plurality of particles 11 may be simultaneously observed by detector 20; in such embodiments, detector 20 may be used to measure an intensity distribution of light emitted by the plural particles 11 in order to characterize the particles 11.

FIGS. 2A-2D each illustrate embodiments in which light receivers 40 include optical fibers as light-transmitting conduits 41, although other structures may be employed as light transmitting conduits. As illustrated in FIGS. 2A-2D, the optical fibers may be tapered, with a greater diameter at its light receiving end than its light emitting end, providing a greater area for receiving light emitted by particle 11, while allowing the light emitting ends to be more densely packed versus embodiments in which non-tapered optical fibers are utilized.

In FIG. 2A, the light emitting ends of the light transmitting conduits 41 output light received from particle 11 to a single common photodetector 45. In such an embodiment, the use of a plurality of light receivers 40 captures light emitted by particle 11 over a greater area than conventional techniques. In some embodiments, photodetector 45 is a photomultiplier; however, those skilled in the art would appreciate that many other types of photodetectors, such as photodiodes, may be employed instead. Characteristics observed by photodetector 45 may relate to scattering, absorption, dispersion, reflection, refraction, diffraction, interference, polarization, coherence, wavelength, spectrum, color temperature, reflectance, or other optical phenomena or characteristics caused light by particle 11. A signal generated by photodetector 45 is provided to computer 60. Computer 60 includes one or more microprocessors programmed to determine a characteristic of the light collected from particle 11. For example, computer 60 may determine intensity, wavelength, and polarization characteristics of the light emitted by particle 11. Based the determined characteristic, computer 60 may determine a characteristic of particle 11. For example, in an embodiment directed to sorting X- and Y-bearing sperm cells, the intensity of fluorescent light emitted by particle 11 may be used to categorize particle 11 as one of an X-bearing sperm cell, a Y-bearing sperm cell, or an indeterminate (uncertain as to whether X- or Y-bearing) sperm cell. Based on the categorization of the particle 11, computer 60 may control a sorting apparatus 70. An example of such a sorting apparatus is described in U.S. Pat. No. 7,371,517, discussed above. However, in some embodiments sorting may not be performed, for example where it is simply desired to analyze a stream of particles. By characterizing particles, the particles may be individually or in aggregations be identified, selected, or graded, for example. A program or programs employed by computer 60 may be stored in a volatile or nonvolatile memory, including RAM, ROM, flash memory, or a hard drive. Also, data calculated by computer 60 may be displayed for review. Additionally, computer 60 may control modulation of laser 30.

By disposing plural light receivers 40 around a point at which particle 11 emits light, light from that particle can be gathered from many more directions compared to the conventional approach with just 0 degree and 90 degree detectors. This can be employed simply to increase the effective aperture of the detection system, or may be used to provide more fine-grained information as to the distribution of fluorescent output.

The light gathered by light receivers 40 may be directed to plural photodetectors such that categorization is determined based upon light gathered at two positions offset by approximately 90 degrees. However, in contrast to the conventional approach in which 0 and 90 degree detectors are at set positions, the disclosed system can, on a particle-by-particle basis, selectively choose a pair of light receivers 40 from the larger population of light receivers 40 for performing characterization. In one example, a large number of light receivers 40 might be split into 360 different groups. Then, based upon the light received by each group, computer 60 would select a pair of groups disposed at 90 degrees from each other and their measurements used for categorizing a particle 11. In this manner, detector 20 is less dependent upon a particular orientation of particle 11 as it passes through detector 20. Ideally, detector 20 could be used for effective characterization of particles, regardless of their orientation as they pass through detector 20. Accordingly, the detector proposed by this invention should more robustly handle variations in the orientation of cells as they pass through the detector, and for embodiments that perform sorting of particles 11, a higher proportion of cells may be accurately characterized for sorting and result in a higher purification yield in the sorted sample.

In FIG. 2B, the light emitting end of each light transmitting conduit supplies light to a separate photodetector 45. In some embodiments, the plurality of light transmitting conduits 41 are divided into a plurality of groups of light transmitting conduits 41, with all of the light transmitting conduits 41 in a given group outputting light to one of photodetectors 45a-45n. For example, one embodiment might have three light transmitting conduits conveying light emitted by particle 11 to a common photodetector 45. However, as shown in FIG. 2B, the plurality of photodetectors 45a-45n may be arranged on a single photodetector array 46, such as a linear CCD detector, or a two-dimensional array of photodiodes. FIG. 2B illustrates light transmitting conduits 41a-41n connected to photodetectors 45a-45n, which are included in a single photodetector array 46. Signals generated by each of photodetectors 45 are provided by photodetector array 46 to computer 60. Computer 60 includes one or more microprocessors programmed to determine a characteristic of the light collected by each of the light transmitting conduits and received by their respective photodetectors 50. For example, computer 60 may be programmed to take into consideration the orientation of each of the light receiving ends of light receivers 40. Also, computer 60 may be programmed to consider the relative intensities of the signals provided by photodetectors 45. Accordingly, computer 60 can determine from which direction the greatest amount of light is being received, and select a signal from one or more photodetectors 45 based on this information in order to determine a characteristic of particle 11. Additionally, where filters and/or prisms are used by the detector 20, as discussed above, computer 60 may also take into consideration wavelength information about the light provided by each of the optical fibers 41.

FIG. 2C illustrates an embodiment similar to FIG. 2B, except with plural lasers 30a and 30b, and plural photodetector arrays 46a and 46b. A plurality of light sources may be employed to more evenly irradiate particle 41 from multiple directions, including from positions outside of the planar cross section illustrated in FIG. 2C. Also, lasers 30a and 30b may be at different wavelengths and/or intensities. Additionally, the lasers can be configured to illuminate particle 11 at differing positions, and, accordingly, at different times, as it passes through detector 20, allowing a plurality of characteristics of a single particle 11 to be detected by detector 20. Characterization of a particle 11 may be performed using any combination of one or more light sources and one or more light receivers selected from among the plurality of light receivers included in detector 20. By using a plurality of photodetector arrays 46a and 46b, the routing and connection of the plurality of optical fibers 41 may be simplified. For example, in FIG. 2B light transmitting conduits 41a and 41n near laser 30 are routed from the far side of detector 20 relative to the position of photodetector array 46. With a large number of light transmitting conduits, such routing may be difficult. In contrast, by employing multiple photodetector arrays 46a and 46b as shown in FIG. 2C, the number of connections to each photodetector array 46 is reduced, and the length of the light transmitting conduits 41 may be reduced.

In FIG. 2D, a plurality of photodetectors 45a-45d is provided, with a plurality of light transmitting conduits 41 associated with each photodetector 45. Additionally, there may be an optical multiplexer 47 provided between the light transmitting conduits and the photodetectors 45, with the optical multiplexer 47 under control of computer 60. This allows computer 60 to selectively choose from which of a plurality of light transmitting conduits 41 light is directed to a given photodetector 45. For example, if the group of three light transmitting conduits 41a-41c corresponds to photodetector 45a, optical multiplexer 47 may allow computer 60 to selectively determine which of light transmitting conduit 41a-41c supplies light to photodetector 45a. Optical link 48 connects optical multiplexer 47 and photodetector 45a. A photodetector array 46 can be used instead of discrete photodetectors 45. The use of optical multiplexer 47 can allow a reduction in the number of photodetectors 45 employed, while still allowing detector 20 to gather light from many different positions around particle 11. Computer 60 is one or more microprocessors programmed to determine a characteristic of the received light, and based upon this a characteristic of particle 11.

FIG. 3 illustrates an embodiment of the disclosed subject matter from a side view, in contrast to the cross-sectional views illustrated in FIGS. 1 and 2A-2D. The illustrated detector 20 includes approximately 140 light receivers 40, with their light receiving ends arranged in a spherical arrangement about the point where particle 11a emits light due to irradiation by laser 30a. Although FIG. 3 illustrates a spherical arrangement, other closed or largely closed three-dimensional shapes may be used, including, but not limited to, an ellipsoid or a box shape. Flow source 10 provides a stream of particles 11 for characterization. An example of a flow source is described in U.S. Pat. No. 7,371,517, which produces a stream of entrained sperm cells for characterization and sorting. In the embodiment illustrated in FIG. 3, the top and bottom are open for passage of the stream of particles 11. At some positions detector 20 may not have a light receiver 40 in order to allow entry of laser light. At other positions detector 20 may not have a light receiver in order to allow exit of laser light. For example, the light receiver array is arranged to provide a gap in the embodiment illustrated in FIG. 3 to allow laser 30a to stimulate particle 11a, and a gap could be similarly provided opposite laser 30a for the exit of laser light that does not strike a particle 11. FIG. 3 also illustrates a second laser 30b, with a laser beam that intercepts the stream of particles 11 at a nonorthogonal angle. Additionally, laser 30b is positioned to irradiate the stream of particles 11 at a different position than that of laser 30a (i.e., at the position of particle 11b rather than particle 11a. Alternatively, laser 30b might be positioned to irradiate the stream of particles 11 at the same position as laser 30a, where particle 11a is located. As discussed above, lasers 30a and 30b may be of different wavelengths and/or intensities, to allow broader characterization of particles passing through detector 20. The number of light receivers 40 may be related to the radius at which they are disposed, the diameter of each of the light receivers 40, and the density at which light receivers 40 are to be packed. After particles 11 have passed through detector 20 and been characterized, they may be sorted by sorter 70, as discussed above with respect to other illustrative embodiments. By use of a spherical detector system, essentially any particle, however oriented when it is irradiated by laser 30a, will have one or more detectors situated so that effective characterization of the particle may be performed, thereby increasing the proportion of particles that is successfully characterized, and, in an example of a flow cytometry-based sorting system for purifying sperm cells, enhance the efficiency of the sorting system.

FIGS. 4A-4C illustrate various example configurations for the light-receiving ends of the light receivers 40. In FIG. 4A, the light receiving ends of light receivers 40 are circular, and arranged in a grid-like pattern, as also illustrated in FIG. 3A. In FIG. 4B, the light receiving ends of light receivers 40 are also circular, but are hexagonally packed to increase the proportion of the area what is covered. In FIG. 4C, the light receiving ends of light receivers 40 are square-shaped, such that there are no gaps between neighboring light receivers 40. This might be accomplished by using square-shaped light-transmitting conduits 41 or square-shaped lenses 43 that provide light to more conventional round optical fibers 41. Those skilled in the art would appreciate that other configurations for light receivers 40 might be employed. Additionally, light receivers 40 may be more sparsely distributed, such that there are significant gaps between light receivers 40.

### Description of a Particular Embodiment

### Sorting Sperm Cells

The sperm cells in a sperm sample are associated with an agent that interacts with the DNA in the sperm cells. Preferably the agent is a membrane permiant, noncytotoxic, supravital DNA specific fluorochrome. The interaction between the agent and DNA, can take place through ionic, covalent or hydrogen bonding, for example, so long as the genetic health of the cell is preserved. Preferably, the more DNA in the cell, the more DNA-interacting agent becomes associated with the cell. Even more preferably, a greater amount of DNA in a sperm cell results in the association with a greater amount of detectable DNA-interacting agent and a corresponding increase in detectable signal, e.g. fluorescence, emanating from the cell. In one embodiment, the detectable DNA-interacting agent is a fluorescent DNA dye that is suitable for use in flow cytometry applications. Preferably, the fluorescent agent is selected from the group consisting of, but not limited to, Hoechst 33342, DAPI, Hoechst 33258, SYTOX Blue, Chromomycin A3, Mithramycin, YOYO-1, SYTOX Green, SYTOX Orange, Ethidium Bromide, 7-AAD, Acridine Orange, TOTO-1, TO-PRO-1, Thiazole Orange, Propidium Iodide (PI), TOTO-3, TO-PRO-3 and LDS 751.

The system performs differentiation of X chromosome and Y chromosome bearing sperm on the basis of differences in DNA content between the two sperm cell classes as indicated by the intensity of the fluorescence from each cell. The sperm cells within a sample are separated by a device that can determine whether the accumulated activity, i.e., activity intensity, of a sperm cell that has been contacted with a detectable DNA-interacting agent, is associated with whether a given sperm cell bears an X or Y chromosome.

Preferably, the activity intensity is an amount of fluorescence directly related to the amount of DNA in the sperm cell. Preferably, a flow cytometry apparatus separates cells that exhibit fluorescence that falls into a given range or window of intensity. Setting up a flow cytometer to separate cells in a particular window of fluorescence is also referred to as “gating” the flow cytometer. Accordingly, the invention envisages calculating a window of fluorescence intensity (range of fluorescence intensity) that is indicative of either X- or Y-bearing sperm. For example, the separation of X- from Y-chromosome bearing sperm is based on the fact that human Y-bearing sperm contain about 2.8% less DNA than X-bearing sperm. If the DNA content of a normal Y-bearing human sperm is assigned an index value of 100, by extension, an X-bearing sperm will have a value of about 102.8. Additionally, the window could be adjusted to take into account differences in DNA between X and Y chromosomes of sperm from different species such as pig, goat, horse, bull, canine, feline, etc.

For small differences in DNA to be detected, the sperm is subjected to flow cytometry. Details of a conventional generalized methodology of sperm flow cytometry are described in U.S. Pat. Nos. 5,985,216 and 5,135,759, which are hereby incorporated by reference in their entirety. Conventionally, sperm cells pass single file through the laser beam, and the DNA content of individual sperm is measured by way of its association with the detectable DNA specific agent. For example, a suspension of single cells stained with a fluorochrome is made to flow in a narrow stream intersecting an excitation source (laser beam). As single cells pass through the beam, an optical detector collects the light emitted by the cells, converts the light to electrical signals, and the electrical signals are analyzed by a programmed computer. Data may be displayed as multi- or single-parameter histograms, using number of cells and fluorescence per cell as the coordinates.

A specially modified orienting nozzle may be used to control the orientation of the flat ovoid sperm head as it passes the laser beam. Conventionally, about 10-15% of sperm nuclei are properly oriented as they pass through the laser beam; the use of a orienting nozzle can result in a significant increase in the percentage of sperm that are properly oriented. For example, in a modified BD Vantage® SE flow cytometer/cell sorter, hydrodynamic forces exerted on the flat, ovoid mammalian sperm nuclei orient the nuclei in the plane of the sample stream as they exit the injection tip. The sample stream is broken into uniform droplets by an ultrasonic transducer. Individual droplets containing single sperm are given a charge and electrostatically deflected into collection vessels based upon their characterization. The collected sperm nuclei then can be used for intrauterine insemination or for microinjection, e.g., by intracytoplasmic sperm injection (ICSI), into eggs. Where the sperm nuclei have no tails, they cannot be used for normal insemination but nonetheless can be characterized.

Accurate measurement of mammalian sperm DNA content using flow cytometry and cell sorting is difficult because the sperm nucleus is highly condensed and flat in shape, which makes stoichiometric staining difficult and causes stained nuclei to have a high index of refraction. These factors contribute to emission of fluorescence preferentially from the edge or thin plane of the sperm nucleus. In most flow cytometers and sorters, the orientation of the sample flow is critical for successfully sorting sperm cells, as fluorescence measurement is most accurate when the fluorescent stain in sperm nuclei is excited and the fluorescence is measured on an axis perpendicular to the plane of the sperm head. At relatively low sample flow rates, hydrodynamics may be used to orient tailless sperm so that DNA content can be measured precisely on about 60 to 80% of the sperm passing in front of the laser beam, as observed with bovine sperm. The preferred modified BD Vantage® system can present tailless sperm from most species at the rate of about 2,000 to about 5,000 sperm per second for characterization. Intact sperm (with tails), whether viable or nonviable, cannot be oriented as effectively as tailless sperm nuclei.

It is, of course, of critical importance to maintain high viability of the intact sperm during the sorting process and during storage after sorting but prior to insemination. Of the factors involved in maintaining sperm viability, the method of staining, the sheath fluid, and the collecting fluid have been found to be especially important.

A nontoxic detectable DNA-interacting agent must be selected. A preferred stain is Hoechst bisbenzimide H 33342 fluorochrome (Calbiochem-Behring Co., La Jolla, Calif.). Preferably, concentration of the fluorochrome is be minimal to avoid toxicity, and yet be sufficient to stain sperm uniformly and to detect the small differences in the DNA of euploid and aneuploid sperm with minimal variation. A suitable concentration was found to be 5 μg/ml, but this may be varied from 4 to 5 μg/ml.

Preferably, the sperm sample is incubated with stain at sufficient temperature and time to allow the stain to associate with the DNA, but under mild enough conditions to preserve sperm viability. Incubation for 1 hr at 37° C. was found to be acceptable, but ranges of 30° to 39° C. would also be effective. Incubation time may be adjusted according to temperature; e.g., 1.5 hr for 30° C.; 1 hr for 39° C.

Sheath fluid used in sorting cells should be electrically conductive and isotonic and compatable with maintaining cell viability. A concentration of about 10 mM phosphate buffered saline provides the preferred electrical properties, and the saline may be supplemented with protein, such as serum albumin, to enhance sperm viability by providing protein support for metabolism and viscosity for the sperm. Preferably, the sheath fluid is free of sugars and excess salts.

Dilution of sperm as occurs in sorting tends to reduce viability of the cells. To overcome this problem, sperm may be collected in test egg yolk extender (TYB) that may be modified by adjusting the pH and/or by adding a surfactant. Details of the composition of the extender are described in U.S. Pat. No. 5,136,759, which is hereby incorporated by reference in its entirety. The surfactant is believed to enhance capacitation of the sperm of some species prior to fertilization.

Example of Sperm Preparation and Staining

Prior to evaluation and processing, freshly collected semen is allowed to liquefy at 35° C. for 30 minutes. Semen may be evaluated for volume, concentration, percentage motile, progression (grade 0-3), and viability (eosin dye exclusion) before and after processing. Semen may be processed to recover motile sperm and to remove undesirable seminal components by discontinuous density gradients (ISolate, 50%, 90%, Irvine Scientific, Santa Ana, Calif.). After processing, recovered sperm are washed and the pellets resuspended in BWW (Irvine Scientific, Santa Ana, Calif.) supplemented with 10% bovine serum albumen (BA; Sigma, St Louis, Mo.), and then stained for 1 hour at 37° C. with Hoechst 33342 (Calbiochem-Behring Corporation, La Jolla, Calif.) at a final concentration of 9 μM as previously described (Johnson et al., Hum. Reprod., 8, 1733-1739, 1993).

Example of Flow Cytometric Separation

Stained sperm may be sorted using, for example, a modified Beckman Coulter Epics® 753 (Beckman Coulter, Inc., Brea, Calif.) or modified FACS® Vantage flow cytometers (Becton-Dickinson Immunocytometry Systems, San Jose, Calif.) equipped with argon ion lasers. Dulbecco's phosphate buffered saline (Irvine Scientific, Santa Anna, Calif.) was used as sheath fluid. Fluorescence emitted by each stained sperm after laser excitation (100 mW UV) may be directed through a 400 nm long pass filter.

In this disclosure there are described only the preferred embodiments of the disclosed subject matter and but a few examples of its versatility. It is to be understood that the disclosed subject matter is capable of use in various other combinations and environments and is capable of changes or modifications within the scope of the inventive concept as expressed herein. Thus, for example, those skilled in the art will recognize, or be able to ascertain, using no more than routine experimentation, numerous equivalents to the specific substances and procedures described herein. Such equivalents are considered to be within the scope of this invention.

What is claimed is:

1. A particle characterization system comprising:
a flow source to produce a stream of particles;
a source of light or other energy directed at the stream of particles to cause fluorescent or scattered light to be emitted from the particles;
a detector of the fluorescent or scattered light comprising a plurality of light receivers in an enclosed three-dimensional arrangement to detect the light emitted by the particles; and
a computer which receives information from the detector regarding light emitted by a particle in response to the source of light or other energy, the computer programmed to determine a characteristic of the particle based on the light collected from the particle.

2. The particle characterization system of claim 1, further comprising:
a sorting system which enables collection of particles exhibiting a desired characteristic.

3. The particle characterization system of claim 1, wherein
the light receivers are arranged in a substantially spherical or ellipsoid shape about a point in the stream of particles at which the light is emitted by the particles.

4. The particle characterization system of claim 3, wherein
each of the light receivers comprises an end of an optical fiber, liquid wave guide, nanofiber, or other light-transmitting conduit which receives the light emitted by the particles.

5. The particle characterization system of claim 4, wherein
each of the light receivers further comprises a lens for collecting the light emitted by the particles and directing the collected light to the respective end of the light-transmitting conduit included in the light receiver.

6. The particle characterization system of claim 1, wherein
the particles are cells to which a fluorescent dye that binds to DNA has been applied.

7. The particle characterization system of claim 6, wherein
the cells are sperm cells.

8. The particle characterization system of claim 6, wherein
the cells are mammalian sperm cells.

9. The particle characterization system of claim 4, wherein
all of the optical fibers receiving the fluorescent light output the received light to a single photodetector.

10. The particle characterization system of claim 4, wherein
the light-transmitting conduit receiving the light emitted by the particle are divided into a plurality of groups of one or more optical fibers, each group of optical fibers outputting their received light to a respective one of a plurality of photo detectors.

11. The particle characterization system of claim 10, wherein
the computer is further programmed to select, based on the light detected by each of the plurality of photodetectors, the measurement of a single photodetector for determining the characteristic of the particle.

12. The particle characterization system of claim 4, further comprising:
a filter or prism to enable collection of light signals at different wavelengths.

13. The particle characterization system of claim 1, wherein
the light receivers are divided into a plurality of groups of one or more light receivers; and
the computer is further programmed to select, based on the amount of light detected by each group, the amount of light received by a single group for determining the characteristic of the particle.

14. The particle characterization system of claim 1, wherein
there are plural sources of light or other energy directed at the stream of entrained particles to cause fluorescent or scattered light to be emitted from the particles.

15. The particle characterization system of claim 1, wherein
the computer is further programmed to characterize the particle based on light emitted by the particle in response to a plurality of sources of light or other energy directed at the stream of particles.

16. The particle characterization system of claim 1, wherein
the computer is further programmed to identify, select, or grade the particle based on the light collected from the particle.
